(19) United States
(12) Patent Application Publication
(10) Pub. No.: US 2007/0154347 A1
(43) Pub. Date: Jul. 5, 2007

(54) LOW TEMPERATURE PROCESS FOR CONCURRENT CLEANING AND SANITATION OF SOLID SURFACES

(76) Inventors: John S. Novak, Naperville, IL (US); Edward F. Steiner, Downers Grove, IL (US); Howard Q. Tran, Chicago, IL (US); Ali O. Kilic, Chicago, IL (US); James T.C. Yuan, Boothwyn, PA (US)

Correspondence Address: AIR LIQUIDE;Intellectual Property, 2700 POST OAK BOULEVARD, SUITE 1800, HOUSTON, TX, 77056, US

(21) Appl. No.: 11/560,270

(22) Filed: Nov. 15, 2006

Related U.S. Application Data
(60) Provisional application No. 60/741,372, filed on Dec. 1, 2005.

(51) Int. Cl. A61L 2/18; A61L 2/025

(52) U.S. Cl. 422/28; 422/37; 422/20

(57) ABSTRACT

An improved process for the concurrent, low temperature cleaning and sanitation of solid surfaces is provided. This process involves introducing a cryogen and an antimicrobial liquid into a vessel, thereby creating an immersion bath. A sterilant is then introduced into this immersion bath. An object to be cleaned and sanitized is then immersed in this immersion bath for a first period of time. Mechanical energy may be introduced into this vessel for a second period of time.

### CROSS REFERENCE TO RELATED APPLICATIONS

This application claims the benefit of U.S. Provisional Application No. 60/741,372 filed Dec. 1, 2005, the entire contents of which are incorporated herein by reference.

### BACKGROUND

Contamination of solid surfaces can pose serious health concerns. Contamination on food industry surfaces may be microbial (such as E. coli or Salmonella typhimurium) or biological residue from the processed food (such as blood or tissue). Contamination on medical industry surfaces may similarly contain residue from the medical procedure (such as saliva or urine).

Many of these surfaces are difficult to effectively clean with conventional methods. It is well known that rough surfaces are more difficult to clean than smooth surfaces. For example, a 1994 study showed that there were living bacteria in biofilms adherent to wood and plastic cutting boards, and suggested that these bacteria were a potential source for household and commercial food contamination (Abrashami Tall, et al., “Bacterial Adherence and Viability on Cutting Board Surfaces,” J Food Safety, Vol. 14:153-172, 1994). Cross contamination can also occur when food passes over a contaminated surface.

Medical and dental instruments are often notoriously difficult to effectively clean, since they typically have very small openings, joints, crevices, valves, and channels. They tend to be ideal places for bacterial and viral growth. (See, for example, U.S. Pat. No. 6,858,181, and U.S. Pat. No. 7,033,511.)

These devices often can not be cleaned and sanitized by conventional techniques.
A national survey of doctors and nurses by Gastroenterology Nursing found endoscope cleaning practices seriously flawed. Three-quarters of the respondents noted that their endoscopes became blocked with “biological debris” at least once a year; some reported 30 to 100 blockages a year. And a 1997 report published in the Archives of Family Medicine discovered that most primary care doctors surveyed botched the disinfection and cleaning of flexible sigmoidoscopes used in colorectal exams.” (Finch, “Unclean Instruments,” Hippocrates 12 (2): 40-47, 1998.)

One could make the association that paint, as a representative viscous polymer, is somewhat analogous to biofilm, and that similar issues surround the removal of paint from solid surfaces. In the past, cryogenic liquid has been used to fracture and debond paint from such surfaces (see U.S. Pat. No. 4,554,025). Likewise, paint coatings have been removed from objects by immersing these objects in cooled condensed gases (such as liquid nitrogen), then subsequently blasted with steel shot (U.S. Pat. No. 5,094,696).

Liquid nitrogen has often been used in these types of paint removal processes, due to its very low cryogenic temperature of −196° C. However, methods of this type, using this type of extreme temperature, can damage or distort parts of delicate instruments. In order to circumvent these problems, it is known to use liquid or supercritical CO₂. While CO₂ in these states have been shown to have good solvency for oils, greases and contaminants, liquid CO₂ fails to effectively remove paint, rust or most adhesives, and is therefore relegated to duty as a solvent and degreaser.

The removal of a biofilm, or bacterial contaminant from the surface of food processing equipment, or medal equipment, involves all the problems involved with removing paint from a surface, coupled with the need to decontaminate the freed residue.

There is a need in the industry for a process that can clean and sanitize solid surfaces in an efficient and efficacious manner.

### SUMMARY

The process in the present application is directed to a method that satisfies the need in society in general for a process that can clean and sanitize solid surfaces in a clean and efficacious manner.

In one aspect of the process in the present application, a low temperature process for concurrent cleaning and sanitation of solid surfaces is provided. This process involves introducing a cryogen and an antimicrobial liquid into a vessel, thereby, creating an immersion bath. A sterilant is then introduced into this immersion bath. An object to be cleaned and sanitized is then immersed in this immersion bath for a first period of time.

In one aspect of the process in the present application, a low temperature process for concurrent cleaning and sanitation of solid surfaces is provided. This process involves introducing a cryogen and an antimicrobial liquid into a vessel, thereby creating an immersion bath. A sterilant is then introduced into this immersion bath. An object to be cleaned and sanitized is then immersed in this immersion bath for a first period of time. Then mechanical energy is introduced into this vessel for a second period of time.

### DESCRIPTION OF PREFERRED EMBODIMENTS

Illustrative embodiments are described below. While the process in the present application is susceptible to various modifications and alternative forms, specific embodiments, thereof, have been shown by way of example in the drawings, and are herein described in detail. It should be understood, however, that the description herein of specific embodiments is not intended to limit the process in the present application to the particular forms disclosed, but on the contrary, the process in the present application is to cover all modifications, equivalents, and alternatives falling within the spirit and scope of the process in the present application, as defined by the appended claims.

It will, of course, be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would, nevertheless, be a routine undertaking for those of ordinary skill in the art, having the benefit of this disclosure.

The following definitions are solely to aid readers and are no narrower than the meaning of the terms as understood by a person skilled in the art.

As used herein, the term “biofilm” refers to biological material adhering to a surface, including adhesion matrices associated with the biological material.

As used herein, the term “microorganisms” refers to any noncellular or unicellular (including colonial) organism. Microorganisms include all prokaryotes. Microorganisms include bacteria (including cyanobacteria), lichens, microfungi, protozoa, virinos, viroids, viruses, and some algae.

As used herein, the term “microbe” is synonymous with microorganism. As used herein, the term “microbe” refers to one or more biological agents, including, without limitation, bacteria, viruses, fungi, spores, molds, yeasts, etc., and combinations thereof.

As used herein, the term “antimicrobial” refers to the ability to inhibit, or control the spread or growth of microbes. As used herein, the term “antimicrobial” refers to the ability of the compounds of the process in the present application to prevent, inhibit, or destroy the growth of microbes, such as bacteria, fungi, protozoa, and viruses.

As used herein, the term “sanitize” refers to use of physical or chemical processes to remove, inactivate, or destroy pathogenic organisms on an object, or its surface, and to render the object safe for handling, use of disposal.

As used herein, the term “sterilant” refers to a physical or chemical agent or process capable of destroying all forms of life (including bacteria, viruses, fungi, and spores) on inanimate surfaces.

As used herein, the term “ambient temperature” refers the nominal temperature of the air (or other gases, liquids, etc.) that surrounds the component, module, assembly, or system.

As used herein, the term “cryogen” refers to any substance having a temperature of or below 0° C.

As used herein, the phrases “medical instrument”, or “dental instrument”, refers to instruments, devices, tools, appliances, apparatus, and equipment used in medicine or dentistry. Such instruments, devices, and equipment can benefit from cleaning and sanitizing process, according to the process in the present application. These various instruments, devices, and equipment include, but are not limited to, diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, endoscopes (e.g., noninvasive flexible and rigid fiber optic endoscopes), endotracheal tubes, anesthesia breathing circuits, cytoscopes, arthoscopes, and related equipment, and the like, or combinations thereof.

As used herein, the phrases “food processing equipment” refers to instruments, devices, tools, appliances, apparatus, and equipment used preparation of fresh products for market, manufacture of prepared food products. Such instruments, devices, and equipment can benefit from cleaning and sanitizing process, according to the process in the present application. These various instruments, devices and equipment include, but are not limited to, bakery machinery, bottling machines, food or drying, can or bottle washing machinery, can making or sealing machinery (food or drying processing), canning machinery, food or drying, cooking equipment (including household), crushing machinery (food processing), distilling equipment, beverage, filters (food processing machinery), flour milling machinery, food packing machinery, food processing or canning machinery, juice extractors, fruit or vegetable (including household), saws, power (food processing), slicing machinery, food, toasters, commercial, electric, and related equipment, and the like, or combinations thereof.

The disclosed embodiments comprise a low temperature process for concurrent cleaning and sanitation of solid surfaces. These solid surfaces may comprise anything that may benefit from the process in the present application, and are specifically directed toward medical instruments, dental instruments, and food processing equipment. The solid surface of interest shall be referred to in this application as the object to be cleaned and sanitized.

One embodiment comprises the introduction of a cryogen and an antimicrobial liquid into a vessel, thus creating an immersion bath. The cryogen may include solid or liquid carbon dioxide, liquid nitrogen, or liquid air. The antimicrobial liquid may include ethanol. Although, for example, ethanol and carbon dioxide will inactivate most vegetative cells of bacterial pathogens, complete sterilization may be achieved only by combining the appropriate proportions of a sterilant for dealing with resistant spores and viruses. This sterilant may include acetic acid, peracetic acid, sodium hydroxide, potassium hydroxide, sodium hypochlorite, quaternary ammonium compounds, hydrogen peroxide, and ozone in any combination or ratio.

The object to be cleaned and sanitized is then immersed into the immersion bath. This object may be immersed for a time sufficient to inactivate any pathogenic microorganisms on the surface, and thus sanitize the object. This first period of time may be between about 1 second and about 10 hours. This first period of time may be between about 5 seconds and about 30 minutes. The immersion bath may have a temperature of between about 0° C. and about −300° C. The immersion bath may have a temperature of between about −50° C. and about −100° C.

In another embodiment, after the introduction of the object to be cleaned and sanitized into the immersion bath, mechanical energy is introduced into the vessel. This mechanical energy may be in the form of vibrational energy, radio waves, vortexing, sound waves, shaking, a sudden impact, or a controlled localized explosion. This mechanical energy will be introduced into the vessel for a time sufficient to inactivate any pathogenic microorganisms on the surface, and, thus, sanitize the object. This second period of time may be between about 1 second and about 60 minutes. This second period of time may be between about 5 seconds and about 5 minutes.

What is claimed is:

1. A low temperature process for concurrent cleaning and sanitation of solid surfaces, comprising:
a) introducing a cryogen and an antimicrobial liquid into a vessel, thereby creating an immersion bath;
b) introducing a sterilant into said vessel; and
c) immerse an object to be cleaned and sanitized into said immersion bath for a first period of time.

2. The process of claim 1, wherein said cryogen comprises solid carbon dioxide.

3. The process of claim 1, wherein said cryogen comprises any fluid that can decrease the ambient temperature.

4. The process of claim 1, wherein said cryogen is selected from the group consisting of:
a) liquid nitrogen;
b) liquid carbon dioxide; and
c) liquid air.

5. The process of claim 1, wherein said antimicrobial liquid is ethanol.

6. The process of claim 1, wherein said sterilant is selected from the group consisting of:
a) acetic acid;
b) peracetic acid;
c) sodium hydroxide;
d) potassium hydroxide;
e) sodium hypochlorite;
f) a quaternary ammonium compound;
g) hydrogen peroxide; and
h) ozone in any combination or ratio.

7. The process of claim 1, further comprising a drying step.

8. The process of claim 1, wherein said first period of time is sufficient to sanitize the object.

9. The process of claim 1, wherein said first period of time is between about 1 second and about 10 hours.

10. The process of claim 1, wherein the first period of time is between about 5 seconds and about 30 minutes.

11. The process of claim 1, wherein said immersion bath has a temperature of between about 0° C. and about −300° C.

12. The process of claim 1, wherein said immersion bath has a temperature of between about −50° C. and about −100° C.

13. A low temperature process for concurrent cleaning and sanitation of solid surfaces, comprising:
a) introducing a cryogen and an antimicrobial liquid into a vessel, thereby creating an immersion bath;
b) introducing a sterilant into said vessel;
c) immersing an object to be cleaned and sanitized into said immersion bath for a first period of time; and
d) introducing mechanical energy into said vessel for a second period of time.

14. The process of claim 13, wherein said cryogen comprises solid carbon dioxide.

15. The process of claim 13, wherein said cryogen comprises any fluid that can decrease the ambient temperature.

16. The process of claim 13, wherein said cryogen is selected from the group consisting of:
a) liquid nitrogen;
b) liquid carbon dioxide; and
c) liquid air.

17. The process of claim 13, wherein said antimicrobial liquid is ethanol.

18. The process of claim 13, wherein said sterilant is selected from the group consisting of:
a) acetic acid;
b) peracetic acid;
c) sodium hydroxide;
d) potassium hydroxide;
e) sodium hypochlorite;
f) a quaternary ammonium compound;
g) hydrogen peroxide; and
h) ozone in any combination or ratio.

19. The process of claim 13, wherein said mechanical energy is in the form selected from the group consisting of:
a) vibrational energy;
b) radio waves;
c) vortexing;
d) sound waves;
e) shaking;
f) sudden impact; and
g) a controlled localized explosion.

20. The process of claim 13, wherein said second period of time is between about 1 second and about 60 minutes.

21. The process of claim 13, wherein said second period of time is between about 5 seconds and 5 minutes.

22. The process of claim 13, further comprising a drying step.

23. The process of claim 13, wherein said first period of time is sufficient to sanitize the object.

24. The process of claim 13, wherein said first period of time is between about 1 second and about 10 hours.

25. The process of claim 13, wherein the first period of time is between about 5 seconds and about 30 minutes.

26. The process of claim 13, wherein said immersion bath has a temperature of between about 0° C. and about −300° C.

27. The process of claim 13, wherein said immersion bath has a temperature of between about −50° C. and about −100° C.
